SEQUENCE LISTING (28 sequences)

SEQ ID NO: 1 - DNA, 23 bp, Artificial Sequence
  Forward HPV GP5+ primer sequence
tttgttactgtggtagatactac

SEQ ID NO: 2 - DNA, 23 bp, Artificial Sequence
  Forward HPV BSGP5+-2 primer sequence
  modified_base (15)..(15): I
tttgttactgttgtagatactac

SEQ ID NO: 3 - DNA, 23 bp, Artificial Sequence
  Forward HPV BSGP5+-3 primer sequence
  modified_base (15)..(15): I
tttgttactgttgtagataccac

SEQ ID NO: 4 - DNA, 23 bp, Artificial Sequence
  Forward HPV BSGP5+-4 primer sequence
  modified_base (15)..(15): I
tttgttacttgtgtagatactac

SEQ ID NO: 5 - DNA, 23 bp, Artificial Sequence
  Forward HPV BSGP5+-5 primer sequence
  modified_base (15)..(15): I
tttttaactgttgtagatactac

SEQ ID NO: 6 - DNA, 23 bp, Artificial Sequence
  Forward HPV BSGP5+-6 primer sequence
tttgttactgtggtagacactac

SEQ ID NO: 7 - DNA, 23 bp, Artificial Sequence
  Forward HPV BSGP5+-7 primer sequence
  modified_base (12)..(12): I
tttgttacagtagtagacactac

SEQ ID NO: 8 - DNA, 23 bp, Artificial Sequence
  Forward HPV BSGP5+-8 primer sequence
  modified_base (12)..(12): I
tttgttacagtagtagataccac

SEQ ID NO: 9 - DNA, 23 bp, Artificial Sequence
  Forward HPV BSGP5+-9 primer sequence
tttgttactgtggtagatactac

SEQ ID NO: 10 - DNA, 41 bp, Artificial Sequence
  Ion Torrent forward sequencing adaptor
ccactacgcctccgctttcctctctatgggcagtcggtgat

SEQ ID NO: 11 - DNA, 25 bp, Artificial Sequence
  Reverse HPV GP6+ primer sequence
gaaaaataaactgtaaatcatattc

SEQ ID NO: 12 - DNA, 25 bp, Artificial Sequence
  Reverse HPV BSGP6+-b primer sequence
gaaaaataaattgtaaatcatactc

SEQ ID NO: 13 - DNA, 25 bp, Artificial Sequence
  Reverse HPV BSGP6+-c primer sequence
gaaaaataaattgcaaatcatattc

SEQ ID NO: 14 - DNA, 11 bp, Artificial Sequence
  barcode sequence 1 (BC1)
gctaaggtaac

SEQ ID NO: 15 - DNA, 11 bp, Artificial Sequence
  barcode sequence 2 (BC2)
gtaaggagaac

SEQ ID NO: 16 - DNA, 11 bp, Artificial Sequence
  barcode sequence 96 (BC96)
gttaagcggtc

SEQ ID NO: 17 - DNA, 29 bp, Artificial Sequence
  Ion Torrent reverse sequencing adaptor
ccatctcatccctgcgtgtctccgactca

SEQ ID NO: 18 - DNA, 65 bp, Artificial Sequence
  Ion-BC1-GP6+
ccatctcatccctgcgtgtctccgactcagctaaggtaacgaaaaataaactgtaaatcatattc

SEQ ID NO: 19 - DNA, 65 bp, Artificial Sequence
  Ion-BC1-BSGP6+-b
ccatctcatccctgcgtgtctccgactcagctaaggtaacgaaaaataaattgtaaatcatactc

SEQ ID NO: 20 - DNA, 65 bp, Artificial Sequence
  Ion-BC1-BSGP6+-c
ccatctcatccctgcgtgtctccgactcagctaaggtaacgaaaaataaattgcaaatcatattc

SEQ ID NO: 21 - DNA, 65 bp, Artificial Sequence
  Ion-BC2-GP6+
ccatctcatccctgcgtgtctccgactcagtaaggagaacgaaaaataaactgtaaatcatattc

SEQ ID NO: 22 - DNA, 65 bp, Artificial Sequence
  Ion-BC2-BSGP6+-b
ccatctcatccctgcgtgtctccgactcagtaaggagaacgaaaaataaattgtaaatcatactc

SEQ ID NO: 23 - DNA, 65 bp, Artificial Sequence
  Ion-BC2-BSGP6+-c
ccatctcatccctgcgtgtctccgactcagtaaggagaacgaaaaataaattgcaaatcatattc

SEQ ID NO: 24 - DNA, 65 bp, Artificial Sequence
  Ion-BC96-GP6+
ccatctcatccctgcgtgtctccgactcagttaagcggtcgaaaaataaactgtaaatcatattc

SEQ ID NO: 25 - DNA, 65 bp, Artificial Sequence
  Ion-BC96-BSGP6+-b
ccatctcatccctgcgtgtctccgactcagttaagcggtcgaaaaataaattgtaaatcatactc

SEQ ID NO: 26 - DNA, 65 bp, Artificial Sequence
  Ion-BC96-BSGP6+-c
ccatctcatccctgcgtgtctccgactcagttaagcggtcgaaaaataaattgcaaatcatattc

SEQ ID NO: 27 - DNA, 20 bp, Artificial Sequence
  beta globin forward primer BGMS3 (F)
aatatatgtgtgcttatttg

SEQ ID NO: 28 - DNA, 20 bp, Artificial Sequence
  beta globin reverse primer BGMS10 (R)
agattagggaaagtattaga

(19) United States
(12) Patent Application Publication
(10) Pub. No.: US 2018/0148780 A1
(43) Pub. Date: May 31, 2018

(54) Next-Generation Sequencing-Based Genotyping Assay for Human Papilloma Virus (HPV)

(71) Applicants: University of Maryland, Baltimore, Baltimore, MD (US); OFFICE OF TECHNOLOGY TRANSFER, NATIONAL INSTITUTES OF HEALTH, Rockville, MD (US)

(72) Inventors: Kevin CULLEN, Bethesda, MD (US); Nicholas AMBULOS, Catonsville, MD (US); Jennifer TROYER, Frederick, MD (US)

(21) Appl. No.: 15/575,977

(22) PCT Filed: May 20, 2016
(86) PCT No.: PCT/US16/33437

§ 371 Date: Nov. 21, 2017

Related U.S. Application Data
(60) Provisional application No. 62/165,306, filed on May 22, 2015.

(51) Int. Cl. C12Q 1/6869; C12Q 1/70; A61P 31/20

(52) U.S. Cl. CPC C12Q 1/6869; C12Q 1/708; G01N 2333/025; C12Q 2535/122; C12N 2710/20021; G01N 2800/26; A61P 31/20

(57) ABSTRACT

Disclosed herein are compositions and methods for detecting and diagnosing HPV in subjects. The method includes using Next Gen Sequencing for detecting HPV in a sample and determining the specific subtype of HPV infection based on the sequencing data.

### CROSS REFERENCE TO RELATED APPLICATIONS

This application claims priority to U.S. Provisional Application No. 62/165,306, filed May 22, 2015 which is hereby incorporated by reference herein in its entirety.

### STATEMENT OF GOVERNMENT SUPPORT

This invention was made with government support under Grant No. CA134274 awarded by the National Institutes of Health. The government has certain rights in the invention.

### BACKGROUND

Head and neck cancer is the sixth most common cancer worldwide (Ferlay et al., Int J Cancer, 2010, 127:2893-2917). The majority of these tumors are squamous cell carcinomas arising from various anatomical sites including the oral cavity, oropharynx, larynx and hypopharynx. Historically, head and neck squamous cell carcinoma (HNSCC) has been associated with alcohol and tobacco use. More recently, infection with human papilloma virus (HPV) has been implicated in the development of HNSCC, predominantly in the oropharynx (D'Souza et al., NEJM, 2007, 356:1944-1956; Gillison et al., JNCI, 2000, 92:709-720). The incidence of HPV associated oropharyngeal cancer has been steadily increasing (Chaturvedi et al., ASCO, 2013, 31:4550-4559). HPV infection has been associated with better clinical outcomes, in part due to better therapeutic response to chemotherapy and radiation (Ang et al., NEJM, 2010, 363:24-35). Therefore, HPV has become an important biomarker in clinical decision-making and prognostication of patients with oropharyngeal SCC.

In addition to cancers of the head and neck, HPV has been associated with cancers of the cervix, penis, anus, and vulva (zur Hausen, Nat Rev Cancer, 2002, 2:342-350). The virus promotes tumorigenesis through integration and transcription of two major viral oncogenes, E6 and E7. These oncogenes inactivate p53 and Rb, respectively, thereby inhibiting apoptosis and driving cell cycle progression (Moody and Laimins, Nat Rev Cancer, 2010, 10:550-560; Scheffner et al., Cell, 1990, 63:1129-1136; Werness et al., Science, 1990, 248:76-79). The E7 protein binds to and degrades Rb, releasing E2F and driving p16 overexpression (Moody and Laimins, Nat Rev Cancer, 2010, 10:550-560). Such overexpression of p16 has been associated with improved outcomes in HNSCC. Twelve HPV genotypes are confirmed to be oncogenic (16, 18, 31, 33, 35, 39, 45, 51, 52, 56, 58, and 59) while several more are currently considered probably (68) and possibly carcinogenic (26, 53, 66, 67, 70, 73, and 82) (IARC Working Group on the Evaluation of Carcinogenic Risks to Humans, IARC Monogr Eval Carcinog Risks Hum, 2007, 90:1-636). HPV 16 has the highest oncogenic potential and has been associated with the majority of both cervical (Monsonego et al., Gynecol Oncol, 2015, 137:47-54) and oropharyngeal cancers (Kreimer et al., AACR, 2005, 14:467-475).

Currently there is no standard screening test for HPV in HNSCC and testing varies between institutions. Moreover, currently available clinical assays may detect HPV but do not type it. Detection strategies measure HPV DNA, HPV RNA, viral E6 and E7 oncoproteins and the downstream cellular target protein, p16 (Westra, Oral Oncol, 2014, 50:771-779). The most commonly used clinical tests are in situ hybridization to detect HPV DNA and p16 immunostaining. In situ hybridization has limited sensitivity and does not indicate the specific HPV type(s) present in an individual biopsy (Rischin et al., J Clin Oncol, 2010, 28:4142-4148; Chernock et al., Arch Otolaryngol Head Neck Surg, 2011, 137:163-169; Mellin et al., Anticancer Res, 2005, 25:4375-4383). p16 immunostaining is highly sensitive, but also fails to indicate the specific HPV types and may be truly discordant with HPV status in a small number of cases (Jordan et al., Am J Surg Pathol, 2012, 36:945-954). Neither of these detection methods distinguishes between high-risk HPV genotypes.

In cervical cancer screening, a few commercial platforms have been FDA approved for high-risk HPV detection in liquid cytology specimens only, however none of these detection methods provides the specific HPV genotype.

To incorporate full HPV genotyping in studies of large cohorts, many investigators have relied on PCR-based strategies where degenerate or pooled primer sets capable of amplifying a significant range of HPV genotypes are paired with hybridization to genotype-specific probes immobilized on beads, membrane arrays or chips (Liu et al., Oral Oncol, 2015, 51:862-869). However, this assay is costly and low throughput.

Accordingly, there is a need for new diagnostic and prognostic methods that permit rapid, sensitive, and accurate typing of HPV genotypes and subtypes in clinical specimens. The present invention fulfills this need.

### SUMMARY

In one embodiment, the invention relates to a method for detecting HPV nucleic acid in a biological sample of a subject suspected of having an HPV infection or an HPV associated disease. In one embodiment, the method comprises the steps of (a) isolating a nucleic acid sample from a biological sample obtained from the subject, (b) determining the nucleic acid sequence of the nucleic acid sample using a next gen sequencing assay, and (c) determining the genotype of HPV in the sample.

In one embodiment, the method of determining the nucleic acid sequence of the nucleic acid sample comprises the steps of (a) contacting the nucleic acid sample with at least one forward PCR primer, (b) contacting the nucleic acid sample with at least one reverse PCR primer, (c) amplifying the HPV nucleic acid using PCR, and (d) sequencing the amplified nucleic acid products using a Next Gen Sequencer.

In one embodiment, the at least one forward PCR primer comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further comprises a nucleic acid sequence for use as a sequencing adaptor. In one embodiment, the at least one forward PCR primer comprises at least two forward PCR primers, wherein each of the at least two forward PCR primers comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further wherein each of the at least two forward PCR primers further comprises a nucleic acid sequence for use as a sequencing adaptor. In one embodiment, the at least one forward PCR primer comprises at least nine forward PCR primers, wherein each of the at least nine forward PCR primers comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further wherein each of the at least two forward PCR primers further comprises a nucleic acid sequence for use as a sequencing adaptor. In one embodiment, the sequencing adaptor comprise or consists of the sequence set forth in SEQ ID NO:10.

In one embodiment, the at least one reverse PCR primer comprises a nucleic acid sequence selected from the group comprising SEQ ID NO:11, SEQ ID NO:12, and SEQ ID NO:13, and wherein each of the at least one reverse PCR primers comprises a nucleic acid sequence for use as a barcode and a nucleic acid sequence for use as a sequencing adaptor. In one embodiment, the at least one reverse PCR primer comprises at least two reverse PCR primers, wherein each of the at least two reverse PCR primers comprises a nucleic acid sequence selected from the group comprising SEQ ID NO:11, SEQ ID NO:12, and SEQ ID NO:13, further wherein each of the at least two reverse PCR primers comprises a nucleic acid sequence for use as a barcode and a nucleic acid sequence for use as a sequencing adaptor. In one embodiment, the at least one reverse PCR primer comprises at least three reverse PCR primers, wherein at least one of the reverse PCR primers comprises the nucleic acid sequence set forth in SEQ ID NO:11, at least one of the PCR primers comprises the nucleic acid sequence set forth in SEQ ID NO:12, and at least one of the PCR primers comprises the nucleic acid sequence set forth in SEQ ID NO:13, further wherein each of the at least one reverse PCR primers comprises a nucleic acid sequence for use as a barcode and a nucleic acid sequence for use as a sequencing adaptor. In one embodiment, the sequencing adaptor comprises or consists of the sequence set forth in SEQ ID NO: 17.

In one embodiment, the method of detecting HPV in the biological sample of the subject comprises aligning a set of sequence data reads to at least one HPV reference sequence, determining the number of sequencing reads that align to the at least one HPV reference sequence, and detecting HPV nucleic acid when at least one sequencing read aligns to the at least one HPV reference sequence in the sample of the subject. In one embodiment, HPV nucleic acid is detected when at least 5,000 sequencing reads align to the at least one HPV reference sequence in the sample of the subject. In one embodiment, at least one of the steps of aligning and determining are performed using a computer system.

In one embodiment, the method of detecting HPV nucleic acid in the subject comprises aligning a set of sequence data reads to at least one HPV reference sequence, identifying sequencing reads that align to the at least one HPV reference sequence, evaluating the nucleic information of the identified sequencing reads to identify at least one HPV subtype-specific genetic variation, and determining the subtype of the HPV based on the at least one HPV subtype-specific genetic variations. In one embodiment, a single HPV subtype is determined for a sample from a subject. In one embodiment, multiple HPV subtypes are determined for a sample from a subject. In one embodiment, multiple HPV subtypes are determined when at least 1% of the sequencing reads for a sample carry a subtype-specific genetic variation associated with each of the HPV subtypes. In one embodiment, at least one of the steps of aligning, identifying, evaluating and determining are performed using a computer system.

In one embodiment, the method of detecting HPV nucleic acid in the subject comprises aligning a set of sequence data reads to at least one HPV reference sequence, identifying sequencing reads that align to the at least one HPV reference sequence, evaluating the nucleic information of the identified sequencing reads to identify at least one HPV genotype. In one embodiment, a single HPV genotype is determined for a sample from a subject. In one embodiment, multiple HPV genotypes are determined for a sample from a subject. In one embodiment, multiple HPV genotypes are determined when at least 1% of the sequencing reads for a sample carry a genotype specific nucleic acid. In one embodiment, at least one of the steps of aligning, identifying, evaluating and determining are performed using a computer system.

In one embodiment, the invention relates to a method for detecting HPV nucleic acid in a biological sample of a subject suspected of having an HPV infection or an HPV associated disease. In one embodiment, the method comprises the steps of (a) isolating a nucleic acid sample from a biological sample obtained from the subject, (b) determining the nucleic acid sequence of the nucleic acid sample using a next gen sequencing assay, (c) determining the genotype of HPV in the sample and diagnosing the subject with infection by HPV.

In one embodiment, the invention further relates to a method for providing a treatment to the subject on the basis of the diagnosis of HPV. In one embodiment, the method of treatment comprises administering an anti-viral agent to the subject. In one embodiment, the method of treatment comprises administering a vaccine to the subject. In one embodiment, the vaccine is specific for the at least one diagnosed HPV genotype of HPV subtype. In one embodiment, the vaccine is specific for at least one HPV genotype of subtype that was not detected in the sample from the subject.

In one embodiment, the invention relates to a composition comprising at least one PCR primer, wherein the at least one PCR primer comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further comprises a nucleic acid sequence for use as a sequencing adaptor. In one embodiment, the nucleic acid sequence for use as a sequencing adaptor consists of the sequence set forth in SEQ ID NO:10.

In one embodiment, the invention relates to a composition comprising at least one PCR primer wherein the at least one PCR primer comprises a nucleic acid sequence selected from the group comprising SEQ ID NO: 11, SEQ ID NO:12 and SEQ ID NO:13 and further comprises a nucleic acid sequence for use as a barcode and a nucleic acid sequence for use as a sequencing adaptor. In one embodiment, the nucleic acid sequence for use as a sequencing adaptor consists of the sequence set forth in SEQ ID NO: 17.

In one embodiment, the invention relates to a composition comprising at least one PCR primer selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further comprising a nucleic acid sequence for use as a sequencing adaptor, and at least one PCR primer selected from the group comprising SEQ ID NO: 11, SEQ ID NO:12 and SEQ ID NO:13 and further comprising a nucleic acid sequence for use as a barcode and a nucleic acid sequence for use as a sequencing adaptor.

In one embodiment, the method relates to a kit comprising at least one PCR primer selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further comprising a nucleic acid sequence for use as a sequencing adaptor, and at least one PCR primer selected from the group comprising SEQ ID NO: 11, SEQ ID NO:12 and SEQ ID NO:13 and further comprising a nucleic acid sequence for use as a barcode and a nucleic acid sequence for use as a sequencing adaptor, for use in the method of detecting HPV nucleic acid in a biological sample of a subject suspected of having an HPV infection or an HPV associated disease. In one embodiment, the kit further comprises control primers. In one embodiment, the control primers have nucleic acid sequences as set forth in SEQ ID NO:27 and SEQ ID NO:28.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 shows tables providing sequence data for the nine forward primers (upper panel) and a representative number of the reverse primers (lower panel) used for generating a pooled, PCR-amplified library for sequencing.

FIG. 2 shows a schematic representation of the Ion Torrent HPV Genotyping Assay. The HPV gene target in each formalin fixed, paraffin embedded (FFPE) tissue DNA sample is amplified using fused primers based on the BSGPS+/6+ primer system (see FIG. 1). Reverse primers append an Ion Adapter and Barcode before the gene-specific sequence. Forward primers append the Ion Adapter only.

FIG. 3 shows a comparison of HPV genotyping of HNSCC and Cervical Carcinomas by a: Roche LINEAR ARRAY HPV Genotyping Kit, b: blinded Ion Torrent Sequencing Assay performed at NCI-FNLCR, c: Ion Torrent Sequencing performed at UMGCC, HPV16-only PCR and p16INK4A IHC. “ND” indicates that sequence analysis was not done.

### DETAILED DESCRIPTION

The present invention relates to a methods and compositions for detecting HPV nucleic acid in a patient sample. In various embodiments, the methods and compositions of the invention are useful for determining the genotype and/or subtype of HPV present in patent sample. Thus, the present invention relates to methods of genotyping HPV in a patient sample, and compositions for use therein. In one embodiment, the method of the invention can be used for diagnosis of an HPV infection in an patient. In one embodiment, the method of the invention can be used to determine a treatment regimen on the basis of the genotype or subtype of the HPV present in the sample.

In a particular embodiment, the invention relates to the use of a set of primers that are designed to amplify sequences that allow identification of a specific genotype and/or subtype of HPV causing infection. In one embodiment, the primers further comprise a sequencing adaptor region which allows the amplified HPV sequences to be utilized in a Next-Gen Sequencing assay. In one embodiment, one or more of the primers contains a barcode region whereby all the amplified sequences for a single sample comprise the same barcode. Therefore, in one embodiment, amplified sequences from multiple samples are pooled together for sequencing and the sequencing reads are then sorted on the basis of the sample-specific barcodes.

In one embodiment, the method of the invention allows for the identification of the presence of nucleic acid from HPV in a sample. In one embodiment, the methods of the invention allow for the identification of the genotype or subtype of HPV present in a sample. In one embodiment, the methods of the invention allow for the identification of nucleic acid from multiple genotypes or subtypes of HPV in a sample.

The invention further relates to a method of treating a subject based on the diagnosis of HPV infection by one or more HPV subtypes or genotypes. In one embodiment, a treatment is administered to a subject to treat an identified HPV subtype or genotype. In one embodiment, a treatment is administered to a subject to prevent infection by one or more HPV subtypes or genotypes that was not detected in a sample from the subject.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Amplification,” as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to polymerase chain reaction (PCR), multiplex PCR, Rolling Circle PCR, ligase chain reaction (LCR) and the like, in a situation where the target is a nucleic acid, an amplification product can be made enzymatically with DNA or RNA polymerases or transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 10-100 “cycles” of denaturation and replication.

“Amplification products,” “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

“Appropriate hybridization conditions” as used herein may mean conditions under which a first nucleic acid sequence (e.g., primer, etc.) will hybridize to a second nucleic acid sequence (e.g., target, etc.), such as, for example, in a complex mixture of nucleic acids. Appropriate hybridization conditions are sequence-dependent and will be different in different circumstances. In one embodiment, an appropriate hybridization conditions may be selective or specific wherein a condition is selected to be about 5-10 lower than the thermal melting point (Tm) for the specific sequence at a defined ionic strength pH. In one embodiment, an appropriate hybridization condition encompasses hybridization that occurs over a range of temperatures from more to less stringent. In one embodiment, a hybridization range may encompass hybridization that occurs from 98° C. to 50° C. According to the invention, such a hybridization range may be used to allow hybridization of the primers of the invention to target sequences with reduced specificity, for the purposes of amplifying a broad range of HPV genotypes with a single set of primers.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Fragment” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence. Thus, a nucleic acid also encompasses a probe that hybridizes under appropriate hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Primer” as used herein refers to a single-stranded oligonucleotide or a single-stranded polynucleotide that is extended by covalent addition of nucleotide monomers during amplification. Nucleic acid amplification often is based on nucleic acid synthesis by a nucleic acid polymerase. Many such polymerases require the presence of a primer that can be extended to initiate such nucleic acid synthesis.

As used herein, “sample” or “test sample,” may refer to any source used to obtain nucleic acids for examination using the compositions and methods of the invention. A test sample is typically anything suspected of containing a target sequence. Test samples can be prepared using methodologies well known in the art such as by obtaining a specimen from an individual and, if necessary, disrupting any cells contained thereby to release genomic nucleic acids. These test samples include biological samples which can be tested by the methods of the present invention described herein and include human and animal cells, tissues and body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas, buccal cells, cervicovaginal cells, epithelial cells from urine, fetal cells, or any cells present in tissue obtained by biopsy and the like; biological fluids such as cell culture supernatants; tissue specimens which may be fixed; and cell specimens which may be fixed.

Any DNA sample may be used in practicing the present invention, including without limitation eukaryotic, prokaryotic and viral DNA. In one embodiment, the target DNA represents a sample of genomic DNA isolated from a patient. This DNA may be obtained from any cell source, tissue source, or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. DNA is extracted from the cell source, tissue source, or body fluid using any of the numerous methods that are standard in the art. It will be understood that the particular method used to extract DNA will depend on the nature of the source.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of about 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under appropriate hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Target” or “target sequence” may refer to nucleic acid sequences to be amplified. These include the original nucleic acid sequence to be amplified, its complementary second strand and either strand of a copy of the original sequence which is produced in the amplification reaction. The target sequence may also be referred to as the template for extension of hybridized amplification primers.

As used herein, “treating a disease or disorder” means to reduce, diminish or eliminate the frequency and/or severity of a sign and/or symptom of a disease or disorder experienced by a subject.

As used herein, “preventing a disease or disorder” means to reduce, diminish or eliminate the frequency and/or severity of the onset of a sign and/or symptom of a disease or disorder experienced by a subject.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under appropriate conditions to the referenced nucleic acid, complement thereof, or a sequences substantially identical thereto.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In one embodiment, the invention is a high throughput Next Gen Sequencing (NGS)-based assay to identify and type HPV in a biological sample. In some embodiments, the assay is used to identify and genotype HPV in FFPE samples from individuals having or suspected of having cancer associated with HPV, including head and neck squamous cell carcinoma (HNSCC) and cervical cancer (CC).

In some embodiments, the assay of the invention uses PCR-based barcoding to allow the pooling of multiple samples and increased throughput. In some embodiments, one or more forward PCR primers consisting of 1) a sequencing adaptor region and 2) a region that targets HPV is utilized in combination with one or more reverse PCR primers consisting of 1) a region that targets HPV, 2) a barcode region, and 3) a sequencing adaptor region. Interrogation of the sample with the combination of PCR primers allows amplification of HPV sequence information that is useful for genotyping HPV in a sample. Further, the PCR amplicon containing the HPV sequence information comprises a sample-specific barcode and sequencing adaptors. Using this high throughput assay, selected samples can be pooled and assayed for the presence of and the genotype of HPV. The assay of the invention has several advantages: i) it allows pooling of multiple samples into a single sequencing run, ii) it allows genotyping of known HPV variants and also the identification of novel HPV variants, and iii) it allows highly sensitive detection of multi-genotype and/or multi-subtype infection.

In some embodiments, the assay of the invention includes the following steps: (a) providing an amount of genomic nucleic acid isolated from a sample; (b) providing at least one forward and at least one reverse primer of the invention; (c) amplifying HPV regions using PCR; (d) preparing a sequencing library from the amplified PCR products; (e) sequencing the library and (f) analyzing the sequencing data to identify the number of sequencing reads from HPV in the sample and the genotype(s) of the HPV present in the sample. The presence of sequencing reads from one or more HPV genotypes or subtypes is an indication of HPV viral infection.

Any sample from which DNA can be isolated can be used in the assay system. Indeed, in certain instances it may be advantageous to use different sample types, e.g., blood, cancer cells, saliva, and FFPE. Preferably the sample is of human origin.

In some embodiments, multiple samples are amplified in parallel and then pooled to generate a high throughput assay. For example, parallel assays may be carried out in a multi-well plate, such as a 96-well plate or a 384 well plate. The number of pooled samples is not necessarily limited as the limiting factors are 1) the number of sequence specific barcodes and 2) the number of sequencing reads desired per sample for a given sequencing platform. Therefore, the method may be extended to include more samples at a cost of reduced sequencing read coverage per sample.

### Biological Sample

The biological sample can be any sample from which genomic nucleic acid can be obtained. In one embodiment, the target DNA represents a sample of genomic DNA isolated from a patient. The biological sample(s) can be prepared using methodologies well known in the art such as by obtaining a specimen from an individual and, if necessary, disrupting any cells contained thereby to release genomic nucleic acids.

Biological samples which can be tested by the methods of the present invention described herein include human cells, tissues and body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas, buccal cells, cervicovaginal cells, epithelial cells from urine, fetal cells, or any cells present in tissue obtained by biopsy and the like; biological fluids such as cell culture supernatants; tissue specimens which may be fixed; and cell specimens which may be fixed.

This DNA may be obtained from any cell source, tissue source, or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at a site of infection or inflammation. DNA is extracted from the cell source, tissue source, or body fluid using any of the numerous methods that are standard in the art. It will be understood that the particular method used to extract DNA will depend on the nature of the source.

In one embodiment, multiple samples are amplified individually using the method of the invention and pooled together prior to sequencing using a Next Gen Sequencing platform. In one embodiment, multiple samples may be from the same type of biological sample (e.g. all FFPE samples). In one embodiment, multiple samples may be from different types of biological samples.

### Nucleic Acid Samples and Preparation

As contemplated herein, the present invention may be used in the analysis of any nucleic acid sample for which next generation sequencing may be applied. For example, the nucleic acid can be from a cultured cell or cells or a patient cell or tissue or bodily fluid sample. The nucleic acid may be isolated using methods generally known to those of skill in the art, including, but are not limited to, the use of genomic DNA prep kits (commercially available from various sources), manually scraping tissue from slides followed by DNA extraction, and the Pinpoint Slide DNA Isolation System (Zymo Research Corp, Irvine, Calif.).

The nucleic acid may be prepared (e.g., library preparation) for massively parallel sequencing in any manner as would be understood by those having ordinary skill in the art. While there are many variations of library preparation, the purpose is to construct nucleic acid fragments of a suitable size for a sequencing instrument and to modify the ends of the sample nucleic acid to work with the chemistry of a selected sequencing process. Depending on application, nucleic acid fragments may be generated having a length of about 100 to about 1000 bases. It should be appreciated that the present invention can accommodate any nucleic acid fragment size range that can be read by a sequencer. This can be achieved by selecting primers such that the resulting PCR product is within the desired range specific for the sequencer and sequencing method desired. For example, in various embodiments a desired PCR fragment size, including barcode and adaptor regions is about 100, 150, 200, 250, 300, 350, 400, 450 or about 500 bp. Both the 5′ and 3′ ends of the PCR products comprise nucleic acid adapters. In various embodiments, these adapters have multiple roles, such as allowing attachment of the specimen strands to a substrate (bead or flow cell) and having a nucleic acid sequence that can be used to initiate the sequencing reaction through hybridization to a sequencing primer. Further, in some embodiments, the PCR products also contain unique sequences (bar-coding) that allow for identification of individual samples in a multiplexed run. The key component of this attachment process is that each individual PCR product is attached to a bead or location on a slide or flow cell. This single PCR fragment can then be further amplified to generate hundreds of identical copies of itself in a clustered region on the bead, flow cell or slide location. These clusters of identical DNA form the product that is sequenced by any one of several next generation sequencing technologies.

The samples can be sequenced using any massively parallel sequencing platform. Non-limiting examples of sequencers include Ion Torrent PGM, Ion Proton, Illumina MiSeq, Illumina HiSeq 2000 or 2500 and the like.

### PCR Primers

In various embodiments, the assay comprises a combination of at least one forward and at least one reverse PCR primer. In some embodiments, a forward primer of the invention comprises at least a sequencing adaptor region and a virus-specific region. In some embodiments, a reverse primer of the invention comprises at least a virus-specific region, a sample barcode region, and a sequencing adaptor region. The sequencing adaptor region allows for hybridization to a NGS-based sequencing platform, such as a bead or flow cell. In one embodiment, a sequencing adaptor region comprises an Ion Torrent specific adaptor sequence. In one embodiment, a sequencing adaptor region comprises an Illumina specific adaptor sequence.

In one embodiment, at least two forward primers are pooled in a single PCR reaction. In various embodiments, at least 2, 3, 4, 5, 6, 7, 8, 9, 10, 20, 30, 40, 50, 100, 200, 300, 400, 500 or more than 500 forward primers are pooled in a single PCR reaction. In one embodiment, the multiple forward primers target multiple genomic regions associated with a single virus. In one embodiment, the multiple forward primers target multiple genomic regions associated with multiple viruses. In an exemplary embodiment, at least 9 forward primers are pooled in a single PCR reaction targeting multiple genomic regions associated with HPV.

In one embodiment, at least two reverse primers are pooled in a single PCR reaction. In various embodiments, at least 2, 3, 4, 5, 6, 7, 8, 9, 10, 20, 30, 40, 50, 100, 200, 300, 400, 500 or more than 500 reverse primers are pooled in a single PCR reaction. In one embodiment, the multiple reverse primers target multiple genomic regions associated with a single disease. In one embodiment, the multiple reverse primers target multiple genomic regions associated with multiple diseases. In an exemplary embodiment, at least 3 reverse primers are pooled in a single PCR reaction targeting multiple genomic regions associated with HPV.

In one embodiment, a combination of PCR primers for use in the assay comprises a combination of multiple forward and multiple reverse primers. In one embodiment, the combination of primers comprises at least 2, 3, 4, 5, 6, 7, 8, 9, 10, 20, 30, 40, 50, 100, 200, 300, 400, 500 or more than 500 forward primers and at least 2, 3, 4, 5, 6, 7, 8, 9, 10, 20, 30, 40, 50, 100, 200, 300, 400, 500 or more than 500 reverse primers, pooled into a single PCR reaction. In an exemplary embodiment, 9 forward primers and 3 reverse primers are pooled in a single PCR reaction targeting multiple genomic regions associated with HPV.

### PCR Primer Sequence Construct

In some embodiments, in the forward PCR primer, the sequencing adaptor region is located 5′ to the disease specific sequence. In one embodiment, the disease is HPV and the forward PCR primer sequence includes at least one HPV specific sequence selected from the group consisting of SEQ ID NO: 1, 2, 3, 4, 5, 6, 7, 8, and 9. In some embodiments, the forward PCR primer sequence can further include the sequencing adaptor sequence set forth in SEQ ID NO:10. In one embodiment, the forward PCR primer sequence comprises the nucleotide sequence of SEQ ID NO:10 linked to the nucleotide sequence of SEQ ID NO:1. In one embodiment, the forward PCR primer sequence comprises the nucleotide sequence of SEQ ID NO:10 linked to the nucleotide sequence of SEQ ID NO:2. In one embodiment, the forward PCR primer sequence comprises the nucleotide sequence of SEQ ID NO:10 linked to the nucleotide sequence of SEQ ID NO:3. In one embodiment, the forward PCR primer sequence comprises the nucleotide sequence of SEQ ID NO:10 linked to the nucleotide sequence of SEQ ID NO:4. In one embodiment, the forward PCR primer sequence comprises the nucleotide sequence of SEQ ID NO:10 linked to the nucleotide sequence of SEQ ID NO:5. In one embodiment, the forward PCR primer sequence comprises the nucleotide sequence of SEQ ID NO:10 linked to the nucleotide sequence of SEQ ID NO:6. In one embodiment, the forward PCR primer sequence comprises the nucleotide sequence of SEQ ID NO:10 linked to the nucleotide sequence of SEQ ID NO:7. In one embodiment, the forward PCR primer sequence comprises the nucleotide sequence of SEQ ID NO:10 linked to the nucleotide sequence of SEQ ID NO:8. In one embodiment, the forward PCR primer sequence comprises the nucleotide sequence of SEQ ID NO:10 linked to the nucleotide sequence of SEQ ID NO:9. In one embodiment, multiple forward PCR primers comprising SEQ ID NO: 10 linked alternatively to one or more of SEQ ID NOs: 1, 2, 3, 4, 5, 6, 7, 8, and 9 are pooled in a single PCR reaction.

In some embodiments, in the reverse PCR primer, the sequencing adaptor region is located 5′ to the sample barcode region which is 5′ to the disease specific region. In one embodiment, the disease is HPV and the reverse PCR primer sequence includes an HPV specific sequence selected from the group consisting of SEQ ID NO: 11, 12, and 13. In one embodiment, all the reverse PCR primers for use in amplification of a single sample or a single PCR reaction have the same barcode sequence. In exemplary embodiments, a sample specific barcode sequence is selected from the group comprising SEQ ID NO: 14, 15 and 16. The reverse PCR primer sequence can further include the sequencing adaptor sequence set forth in SEQ ID NO:17. Therefore, in one embodiment, one or more reverse sequencing primers for use in a single PCR amplification or with a single sample comprise one or more of SEQ ID NO: 18 (SEQ ID NO:17 linked to SEQ ID NO: 14 linked to SEQ ID NO:11), SEQ ID NO: 19 (SEQ ID NO:17 linked to SEQ ID NO: 14 linked to SEQ ID NO:12), and SEQ ID NO: 20 (SEQ ID NO:17 linked to SEQ ID NO: 14 linked to SEQ ID NO:13). In an alternative embodiment, one or more reverse sequencing primers for use in a single PCR amplification or with a single sample comprise one or more of SEQ ID NO: 21 (SEQ ID NO:17 linked to SEQ ID NO: 15 linked to SEQ ID NO:11), SEQ ID NO: 22 (SEQ ID NO:17 linked to SEQ ID NO: 15 linked to SEQ ID NO:12), and SEQ ID NO: 23 (SEQ ID NO:17 linked to SEQ ID NO: 15 linked to SEQ ID NO:13). In yet a third embodiment, one or more reverse sequencing primers for use in a single PCR amplification or with a single sample comprise one or more of SEQ ID NO: 24 (SEQ ID NO:17 linked to SEQ ID NO: 16 linked to SEQ ID NO:11), SEQ ID NO: 25 (SEQ ID NO:17 linked to SEQ ID NO: 16 linked to SEQ ID NO:12), and SEQ ID NO: 26 (SEQ ID NO:17 linked to SEQ ID NO: 16 linked to SEQ ID NO:13).

In some embodiments, the method provides a number of PCR products that can be used to diagnose a disease or disorder in the subject, or otherwise characterize a biological sample. The number of PCR products used can be between about 1 and about 500; for example about 1-500, 1-400, 1-300, 1-200, 1-100, 1-50, 1-25, 1-10, 10-500, 10-400, 10-300, 10-200, 10-100, 10-50, 10-25, 25-500, 25-400, 25-300, 25-200, 25-100, 25-50, 50-500, 50-400, 50-300, 50-200, 50-100, 100-500, 100-400, 100-300, 100-200, 200-500, 200-400, 200-300, 300-500, 300-400, 400-500, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or any included range or integer. For example, at least about 1, 2, 3, 4, 5, 6, 7, 8, 9, 10, 15, 20, 25, 30, 33, 35, 38, 40, 43, 45, 48, 50, 53, 58, 63, 65, 68, 100, 120, 140, 142, 145, 147, 150, 152, 157, 160, 162, 167, 175, 180, 185, 190, 195, 200, 300, 400, 500 or more total PCR products can be used. The number of PCR products used can be less than or equal to about 1, 2, 3, 4, 5, 6, 7, 8, 9, 10, 15, 20, 25, 30, 33, 35, 38, 40, 43, 45, 48, 50, 53, 58, 63, 65, 68, 100, 120, 140, 142, 145, 147, 150, 152, 157, 160, 162, 167, 175, 180, 185, 190, 195, 200, 300, 400, 500, or more.

### Method of Genotyping

As contemplated herein, the present invention includes methods of genotyping an infectious agent, such as HPV, in a biological sample from Next Gen Sequencing data. Generally, sequence reads are aligned, or mapped, to a reference sequence using, for example, available commercial software or open source freeware (e.g., nucleotide and quality data input, mapped reads output). This may include preparation of read data for processing using format conversion tools and optional quality and artifact removal filters before passing the read data to an alignment tool. Next, variants are called (e.g., summarized data input, variant calls output) and interpreted (e.g., variant calls input, genotype information output).

Standard approaches to mapping and analysis of this type of massively parallel sequence data are applicable to the invention described herein. In some embodiments, an analytical pipeline may detect sequence variation and determine the genotype of an infectious agent, such as HPV, as outlined in the method below. First, raw read data, which may include sequence and quality information from the sequencing hardware, is received and entered into the system. The data is optionally prefiltered, for example, one read at a time or in parallel, to remove data that is too low in quality, typically by end trimming or rejection. For a multiplexed sequencing reaction, the raw reads are sorted according to the barcode region to group reads from each individual sample. The reads are then trimmed to remove barcode and adaptor sequences.

The remaining data is then aligned using a set of reference sequences. Read data can be mapped to reference sequences using any mapping software, and using appropriate alignment and sensitivity settings suitable for the goal of the project. Mapped reads may optionally be postfiltered to remove low quality or uncertain mappings. The total numbers of aligned reads can be determined using any appropriate method including, but not limited to, SAMtools, a PERL script, a PYTHON script, and a sequencing analysis pipeline.

In various embodiments, at least 1000, at least 2000, at least 3000, at least 4000, at least 5000, at least 10,000, at least 50,000, at least 100,000, at least 500,000 or more than 500,000 sequencing reads are determined to be ‘high quality’ after passing quality filters. In one embodiment, ‘high quality’ sequencing reads are aligned to one or more reference sequences.

In one embodiment, sequencing reads are aligned to multiple reference sequences representing different genotypes and/or subtypes of HPV. In one embodiment, sequencing reads are aligned to a reference sequence representing a single genotype and/or subtype of HPV and then the aligned reads are subsequently analyzed for genotypic differences including subtype-specific variants. Subtype specific variants include, but are not limited to, sub-type specific single nucleotide polymorphisms (SNPs), sub-type specific insertions, sub-type specific deletions, sub-type specific microsatellite variations, or any other genetic alteration that can be used to distinguish one subtype of HPV from another.

### Methods of Diagnosing HPV

In one embodiment, at least 1000, at least 2000, at least 3000, at least 4000, at least 5000, at least 6,000, at least 7,000, at least 8,000, at least 9,000, at least 10,000, at least 15,000, at least 20,000, at least 25,000 or more than 25,000 sequencing reads align to one or more HPV reference sequences. In one embodiment, a minimum quality score for alignment is AQ17. In an exemplary embodiment, an infection with HPV is determined when at least 5,000 high quality sequencing reads align to one or more HPV reference sequences.

In one embodiment, less than 5000, less than 4000, less than 3000, less than 2000, less than 1000, less than 500, or less than 100 sequencing reads align to one or more HPV reference sequences. A sample wherein less than 5000 sequencing reads align to one or more HPV reference sequences, but also wherein there was amplification using control primers, is determined to be HPV negative. In one embodiment, control primers are for beta globin positive amplification. In one non-limiting example, control primers are BGMS3 (F) AAT ATA TGT GTG CTT ATT TG (SEQ ID NO:27) and BGMS10 (R) AGA TTA GGG AAA GTA TTA GA (SEQ ID NO:28).

In one embodiment, greater than or equal to 99% of the sequencing reads from HPV associated with a sample are from a single genotype and/or subtype of HPV. Such a sample is then determined to be singly infected with the identified genotype and/or subtype of HPV. In one embodiment, a genotype and/or subtype of HPV is selected from the group comprising HPV1, HPV2, HPV3, HPV4, HPV6, HPV7, HPV8, HPV10, HPV11, HPV13, HPV16, HPV18, HPV22, HPV26, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, HPV82, or another subtype of HPV.

In one embodiment, greater than about 1% of the sequencing reads from HPV associated with a sample are from each of two genotypes and/or subtypes of HPV. In one embodiment, about 1.1% of the sequencing reads are from one HPV genotype and/or subtype and about 98.9% are from a second HPV genotype and/or subtype. In one embodiment, about 2% of the sequencing reads are from one HPV genotype and/or subtype and about 98.0% are from a second HPV genotype and/or subtype. In one embodiment, about 3% of the sequencing reads are from one HPV genotype and/or subtype and about 97% are from a second HPV genotype and/or subtype. In one embodiment, about 5% of the sequencing reads are from one HPV genotype and/or subtype and about 95% are from a second HPV genotype and/or subtype. In one embodiment, about 10% of the sequencing reads are from one HPV genotype and/or subtype and about 90% are from a second HPV genotype and/or subtype. In one embodiment, about 20% of the sequencing reads are from one HPV genotype and/or subtype and about 80% are from a second HPV genotype and/or subtype. In one embodiment, about 30% of the sequencing reads are from one HPV genotype and/or subtype and about 70% are from a second HPV genotype and/or subtype. In one embodiment, about 40% of the sequencing reads are from one HPV genotype and/or subtype and about 60% are from a second HPV genotype and/or subtype. In one embodiment, about 50% of the sequencing reads are from one HPV genotype and/or subtype and about 50% are from a second HPV genotype and/or subtype. Such a sample will be determined to be multiply infected with the identified genotypes and/or subtypes of HPV. In one embodiment, the two or more genotypes and/or subtypes of HPV are selected from the group comprising HPV1, HPV2, HPV3, HPV4, HPV6, HPV7, HPV8, HPV10, HPV11, HPV13, HPV16, HPV18, HPV22, HPV26, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, HPV82, or another genotype and/or subtype of HPV.

In one embodiment, greater than 1% of the sequencing reads from HPV associated with a sample are from each of more than two genotypes and/or subtypes of HPV. Such a sample is determined to be multiply infected with as many HPV genotypes and/or subtypes as are represented as having a fraction of the total HPV associated sequencing reads of over 1%.

In one embodiment, greater than about 1%, greater than about 2%, greater than about 5%, greater than about 10%, greater than about 20%, greater than about 30%, greater than about 40%, greater than about 50%, greater than about 60%, greater than about 70%, greater than about 80%, greater than about 90%, greater than about 95%, greater than about 98%, or greater than about 99% of the sequencing reads from HPV associated with a sample will be from an undetermined HPV genotype and/or subtype. Such a sample will be determined to be infected with at least one unknown or possibly novel genotype and/or subtype of HPV.

### Kits

In one embodiment, the invention provides a kit for use in genotyping HPV in one or more samples. In various embodiments, the kit comprises one of more of: 1) at least one composition comprising pooled forward primers, 2) at least one composition comprising pooled reverse primers wherein each pooled set of reverse primers comprises the same barcode, 3) additional materials and reagents for use in PCR amplification, 4) additional materials and reagents for use in library preparation, 5) positive and negative controls, which may include one or more of control HPV DNA and PCR primers to a control locus, and 6) instructional material describing the use of the kit components.

In various embodiments, a kit of the invention may comprise at least 2, at least 8, at least 16, at least 48, at least 94, at least 384 or more than 384 compositions comprising pooled reverse primers. In one embodiment, the multiple compositions comprising forward and reverse primers are provided in solution. In one embodiment, the multiple compositions comprising forward and reverse primers are provided in powder form. In one embodiment, the multiple compositions comprising forward and reverse primers are provided in individual tubes. In one embodiment, the multiple compositions comprising forward and reverse primers are provided pre-aliquoted in a multi-well plate.

In one embodiment, a pool of forward primers may comprise two or more primers wherein the nucleic acid sequence of the primers comprises a sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9. In one embodiment, a pool of forward primers further comprises one or more control primers. In one embodiment, a control primer has the nucleic acid sequence as set forth in SEQ ID NO:27.

In one embodiment, a pool of reverse primers may comprise three or more primers wherein the nucleic acid sequence of the primers comprises a sequence selected from SEQ ID NO:11, SEQ ID NO:12, and SEQ ID NO:13. In one embodiment, a pool of reverse primers further comprises one or more control primers. In one embodiment, a control primer has the nucleic acid sequence as set forth in SEQ ID NO:28.

In various embodiments, the kit may comprise pooled forward PCR primers at quantities of from about 1 nanogram to 100 milligrams; about 1 microgram to about 10 milligrams; or preferably about 0.1 microgram to about 10 milligrams; or more preferably about 0.1 microgram to about 100 micrograms. In some embodiments, a composition according to the present invention comprises about 5 nanogram to about 1000 micrograms of pooled forward PCR primers. In some embodiments, a composition can contain about 10 nanograms to about 800 micrograms of pooled forward PCR primers. In some embodiments, the composition can contain about 0.1 to about 500 micrograms of pooled forward PCR primers. In some embodiments, the composition can contain about 1 to about 350 micrograms of pooled forward PCR primers. In some embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of pooled forward PCR primers.

In some embodiments, the kit may comprise pooled reverse PCR primers at quantities of from about 1 nanogram to 100 milligrams; about 1 microgram to about 10 milligrams; or preferably about 0.1 microgram to about 10 milligrams; or more preferably about 0.1 microgram to about 100 micrograms. In some embodiments, a composition according to the present invention comprises about 5 nanogram to about 1000 micrograms of pooled reverse PCR primers. In some embodiments, a composition can contain about 10 nanograms to about 800 micrograms of pooled reverse PCR primers. In some embodiments, the composition can contain about 0.1 to about 500 micrograms of pooled reverse PCR primers. In some embodiments, the composition can contain about 1 to about 350 micrograms of pooled reverse PCR primers. In some embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of pooled reverse PCR primers.

### Methods of Treatment

In various embodiments, the invention includes methods and compositions for identifying, classifying, or characterizing samples to diagnose a disease or disorder of a subject from a biological sample obtained from the subject. In one embodiment, the disease or disorder is HPV. In another embodiment, the disease or disorder is cancer associated with HPV, such as, head and neck squamous cell carcinoma (HNSCC) and cervical cancer (CC). In one embodiment, the disease or disorder is an infection with one or more than one HPV genotype and/or subtype. The biological sample can be a cancer sample; for example, the biological sample can be a FFPE biopsy sample of cervical tissue. The methods and compositions disclosed herein can be used to categorize biological samples as originating from a subject that is positive or negative for HPV infection. The methods and compositions disclosed herein can be used to determine or diagnose which genotype and/or subtype of HPV a cancer sample is associated with. The HPV disease status can be used, for example, to decide upon a course of treatment for the cancer.

In some cases, the biological sample is classified as HPV positive for a genotype and/or subtype of HPV with an accuracy of greater than about 75%, 80%, 85%, 86%, 87%, 88%, 89%, 90%, 91%, 92%, 93%, 94%, 95%, 96%, 97%, 98%, 99%, or 99.5%. The classification accuracy as used herein includes specificity, sensitivity, positive predictive value, negative predictive value, and/or false discovery rate.

Also provided herein is a method of treating, protecting against, and/or preventing a disease or disorder in a subject in need thereof. In one embodiment, the method comprises determining one or more HPV genotypes associated with a sample using the method of genotyping of the invention, diagnosing the subject from whom the sample was taken as having an HPV infection of the one or more genotype and/or subtype associated with the one or more HPV genotype, and providing a treatment to the subject on the basis of the one or more identified HPV genotype. In one embodiment, a treatment is an antiviral agent. In another embodiment, the treatment is an anti-cancer agent.

According to some embodiments, a vaccine is delivered to an individual to modulate the activity of the individual's immune system and thereby enhance the immune response against HPV. In some embodiments, the vaccine is selected from the group consisting of: one or more DNA vaccines, one or more recombinant vaccines, one or more protein subunit vaccines, one or more attenuated vaccines and one or more killed vaccines. In one embodiment, the vaccine is specific for the one or more diagnosed HPV genotype and/or subtype. In one embodiment, the vaccine is used to prevent infection by an HPV genotype and/or subtype that was not identified in the sample, and therefore the vaccine is specific to one or more HPV genotype and/or subtype that was not identified.

Routes of administration of a treatment include, but are not limited to, intramuscular, intranasally, intraperitoneal, intradermal, subcutaneous, intravenous, intraarterially, intraocularly and oral as well as topically, transdermally, by inhalation or suppository or to mucosal tissue such as by lavage to vaginal, rectal, urethral, buccal and sublingual tissue. In one embodiment, a vaccine can be administered by means including, but not limited to, electroporation methods and devices, traditional syringes, needleless injection devices, or “microprojectile bombardment gene guns”.

### EXPERIMENTAL EXAMPLE

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

There is currently a need for a sensitive, specific HPV genotyping assay which is rapid, cost effective and appropriate for large sample sets. Such an assay could have great utility both for population studies and for routine diagnosis of HPV-associated cancers in a clinical setting. Current HPV vaccines protect against a limited number of HPV genotypes. The most common HPV genotypes in oropharyngeal cancer are 16, 18, 33, 35 and 58 (Ndiaye et al., The Lancet, 2014, 15:1319-1331), and in the cohort of 185 oropharyngeal cancers tested by the NGS assay, genotype 35 was the most common non-16 genotype (2 cases) (Liu et al., Oral Oncol, 2015, 51:862-869). Notably, HPV 35 is not among the genotypes protected against by the 9-valent Gardasil vaccine. Description and detection of HPV genotypes, subtypes and variants prevalent in different cancer types and within different regions and populations worldwide is an ongoing endeavor better served by sequencing, where the technique allows identification of previously unknown sequence variation, than by hybridization-based techniques which fail to provide readout of the actual sequence present.

Studies examining HPV genotype in HNSCC have commonly relied on post-PCR hybridization methods such as line-blot assays (Liu et al., Oral Oncol, 2015, 51:862-869). While the LAHPV test is somewhat sensitive and specific, it is very costly on a per sample basis as well as labor intensive, making it impractical for many large studies. It has the advantage of comparatively modest up-front equipment costs, but it is not approved for routine diagnostic use in the United States.

Any HPV detection and/or genotyping method relies on the ability to amplify all relevant HPV genotypes. The NGS-based HPV test is based on a pool of primers, BSGPS+/6+, which compared to the formerly standard GPS+/6+ PCR was shown to match or significantly improve amplification of 24 HPV genotypes, displays improved primer alignment to 50 types, and in a cohort of 1,085 clinical samples detected 26 HPV genotypes (Coutlée et al., Clin Microbiol, 206, 44:1998-2006). The LAHPV test is based on the PGMY 09/11 primer pool and is reported by the manufacturer to detect 37 HPV genotypes. The significant agreement between the NGS based test and LAHPV as well as the detection of 20 genotypes in a set of cervical cancer specimens suggest that the primer system used is fully capable of robust amplification of a broad spectrum of HPV genotypes.

The NGS assay presented herein is robust, returning excellent read numbers from FFPE samples. It is highly sensitive and specific and capable of identifying multiple, co-infected HPV genotypes in the same sample. Identification of co-infection by HPV16 by sequence methods, and identification of an HPV16 positive cervical cancer sample, all of which were not identified by LAHPV, demonstrate the increased sensitivity of NGS sequencing. These data also confirm the ability of LAHPV to give false-negative results. Most importantly, the assay is reproducible across laboratories. Of the discordant samples identified between the LAHPV method and NGS sequencing at two independent sites, all but one sample can be explained either as a contaminant (two samples sequenced at NCI-FNLCR) or due to the increased sensitivity of the NGS sequencing approach. When the discrepancies between the different platforms and sites were accounted for, there was essentially a near perfect concordance across the data. Additionally, the data correlate well with direct genotype-specific PCR and p16 IHC assays. Barcoding of the primer sets would permit larger runs of 96 to 384 samples simultaneously with rapid turnaround time and modest technician time. While the NGS assay has a significant capital equipment cost, the per sample reagent cost is less than one tenth of the cost associated with the LAHPV test. As such, a single laboratory could readily support multiple epidemiologic studies or a number of clinical institutions. This study demonstrates the ability of this NGS sequencing assay as an extremely sensitive and accurate method to genotype HPV, which may become the assay of choice for diagnostic laboratories.

The advancement of next generation sequencing technology allows the development of a high throughput, affordable assay for HPV genotyping. The current invention provides an NGS HPV genotyping assay which has been developed using an established primer system with the capacity to detect the broadest range of HPV genotypes with minimal input DNA (≤10 ng), making this amenable for FFPE samples. To evaluate the ability of the assay to accurately genotype HPV in archived FFPE clinical samples, HNSCC and cervical carcinoma samples were genotyped and the genotyping was compared to genotyping by the LAHPV assay. To validate the assay against a wide spectrum of HPV genotypes, 266 cervical cancers from a separate cohort were genotyped.

The materials and methods of this Example are now described.

### Samples

Genomic DNA from 29 oropharyngeal HNSCC samples was prepared from FFPE tissue using the QIAamp DNA FFPE Tissue Kit (Zandberg et al., Cancer Prev Res (Phila), 2015, 8:12-19). In addition, DNA was extracted from FFPE tissue of 13 de-identified cervical cancer cases also using the QIAamp DNA FFPE Tissue Kit. Following IRB approval, all tumor samples were obtained from the University of Maryland Greenebaum Cancer Center (UMGCC) Pathology and Biorepository shared service.

266 cervical cancer specimens from a separate cohort (Lou et al., Clin Cancer Res, 2015, 21:5360-5370) were provided in accordance with the ethical approvals cited. Briefly, DNA was extracted from cervical cancer tissues (5-10 mg) stored in RNAlater using the AllPrep DNA/RNA Micro Kit (QIAGEN) as directed by the manufacturer.

### HPV16 PCR

PCR for the E6 and E7 genes of HPV16 was performed on the HNSCC cases for a previous study (Zandberg et al., Cancer Prev Res (Phila), 2015, 8:12-19). DNA was extracted from several (3 to 5) 10-micron sections of FFPE oropharyngeal cancer tissue using the QIAamp DNA FFPE Tissue Kit (Qiagen) according to the manufacturer's protocol. DNA was quantified using the Quant-iT dsDNA Assay Kit, High Sensitivity (Invitrogen) and stored at −80° C. in aliquots.

p16^{INK4a} Immunohistochemistry

p16^{INK4a} immunohistochemistry was performed on these samples for a previous study (Liu et al., Oral Oncol, 2015, 51:862-869). Briefly, p16 IHC was performed using commercially available antibodies (clone JC8, Santa Cruz Biotechnologies, California) and scored for cytoplasmic and nuclear staining by a consensus of two blinded pathologists. Only tumor cells with moderate or high intensity were counted. Proportional scoring was semi-quantified as follows: 0, <10% staining; 1+, 10-49% staining; 2+, 50-70% staining; 3+, >70% staining. Scores of 2 or 3+ were defined as positive.

### Roche LINEAR ARRAY HPV Genotyping Test®

The protocol is designed to be performed starting with cervical cancer cells collected in preservative media and therefore begins with DNA sample preparation instructions for that starting material. However, for direct comparison to the samples used in the NGS assay, these steps were replaced by the DNA preparation method described above. The manufacturer's protocol was followed beginning with the amplification step. For each sample, 100-500 ng DNA in 50 μl was included in the amplification reaction and tested along with the manufacturer's positive and negative controls (Roche Diagnostics, Indianapolis, Ind.). The PCR machine specified by the protocol was used (96-well GeneAmp PCR System 9700 (Applied Biosystems), however the silver sample block used was not gold-plated. For dilution series, the indicated quantity of DNA was used in the assay.

### Ion Torrent HPV Genotyping Assay

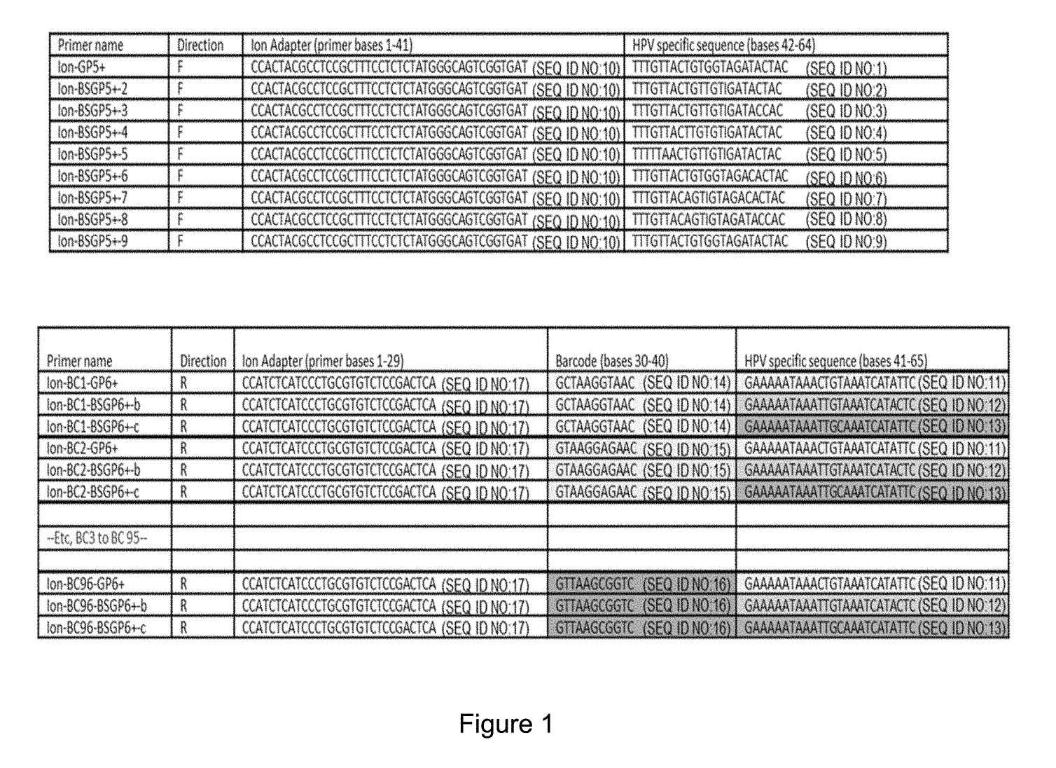

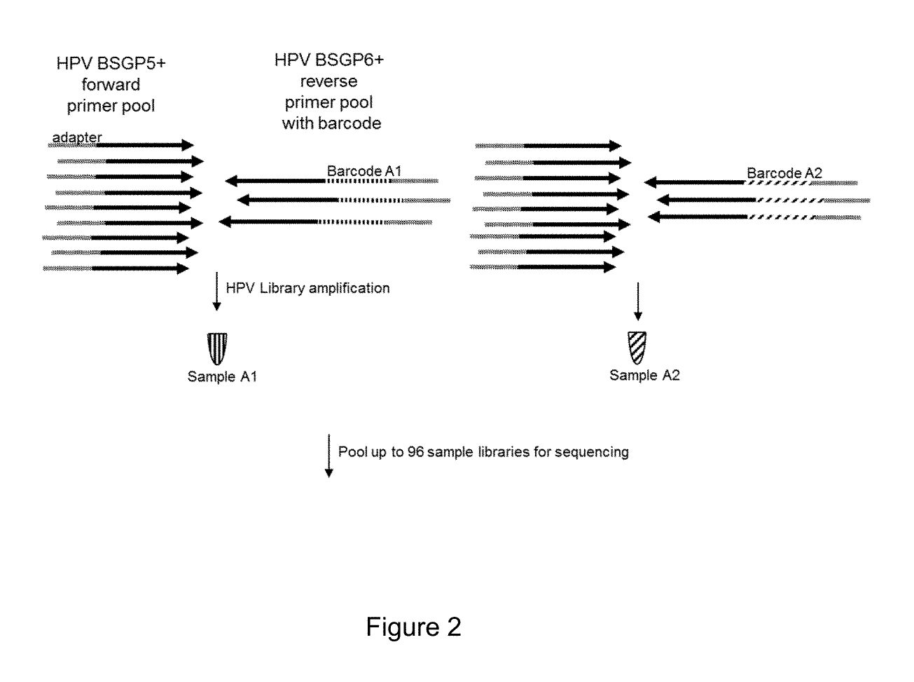

The assay uses the BSGPS+/6+ primer system, which was designed by Schmitt et al. to homogeneously amplify a broad range of HPV genotypes (Schmitt et al., J Clin Microbiol, 2008, 46:1050-1059) and which consists of a pool of 3 reverse primers and 9 forward primers. For use in the Ion Torrent system, the reverse primer sequences are modified by a preceding adapter sequence and one of 96 barcode sequences (FIG. 1), while the forward primer sequences are preceded by adapter sequence only. For each sample, a pool of 3 reverse primers with the same barcode is combined with the forward primer pool to amplify a library of barcoded HPV amplicons. The uniquely identified libraries are then pooled for emulsion PCR and sequencing (FIG. 2).

The assay was initially tested at NCI-Frederick National Laboratory for Cancer Research (NCI-FNLCR). Investigators there were provided with the 29 HNSCC and 8 of the cervical carcinoma DNA samples completely blinded to LAHPV genotyping results. The identical Ion Torrent HPV Genotyping Assay was used in the Genomics Shared Service at the University of Maryland Greenebaum Cancer Center (UMGCC) to analyze the same samples along with 5 additional cervical cancer specimens. Ten ng genomic DNA as quantified by NanoDrop was included in each HPV library amplification reaction. For dilution series, the indicated quantity was included in the reaction.

Library amplification reactions were analyzed using an Agilent BioAnalyzer for presence of product of the expected size (with adapter sequences, ˜150 bp). The investigators at NCI-FNLCR included only samples with an amplified product in the sequencing pool. The investigators at UMGCC included all samples in the sequencing pool at a standardized concentration of approximately 500 pM as determined by the BioAnalyzer. Samples without library product detection were included in the pool at equal volumes. Pooled samples were quantified for emulsion template preparation on the Qubit 2.0 fluorimeter and prepared using Ion PGM 200 bp kits on the One Touch 2. Sequencing was performed on the Ion Torrent PGM utilizing the 200 v2 sequencing chemistry and 316v2 chips.

Raw data collection and processing was performed by the Ion Torrent Server v4.4.3 and mapped to the full-genomic sequences of HPV downloaded from the PAVE database with a minimum quality score of AQ17. Further filtering of only reads greater than 100 bp was performed using NGSutils (Breese and Liu, Bioinformatics, 2013, 29:494-496). A sample must contain more than 5,000 reads in any HPV genotype to be called positive. If a co-infection of HPV is present, the minor number of reads must total greater than 1% of the total number of reads for that given sample. A sample with no reads and beta globin positive amplification using the primers BGMS3 (F) AAT ATA TGT GTG CTT ATT TG (SEQ ID NO:27) and BGMS10 (R) AGA TTA GGG AAA GTA TTA GA (SEQ ID NO:28) was called HPV negative.

The results of this Example are now described.

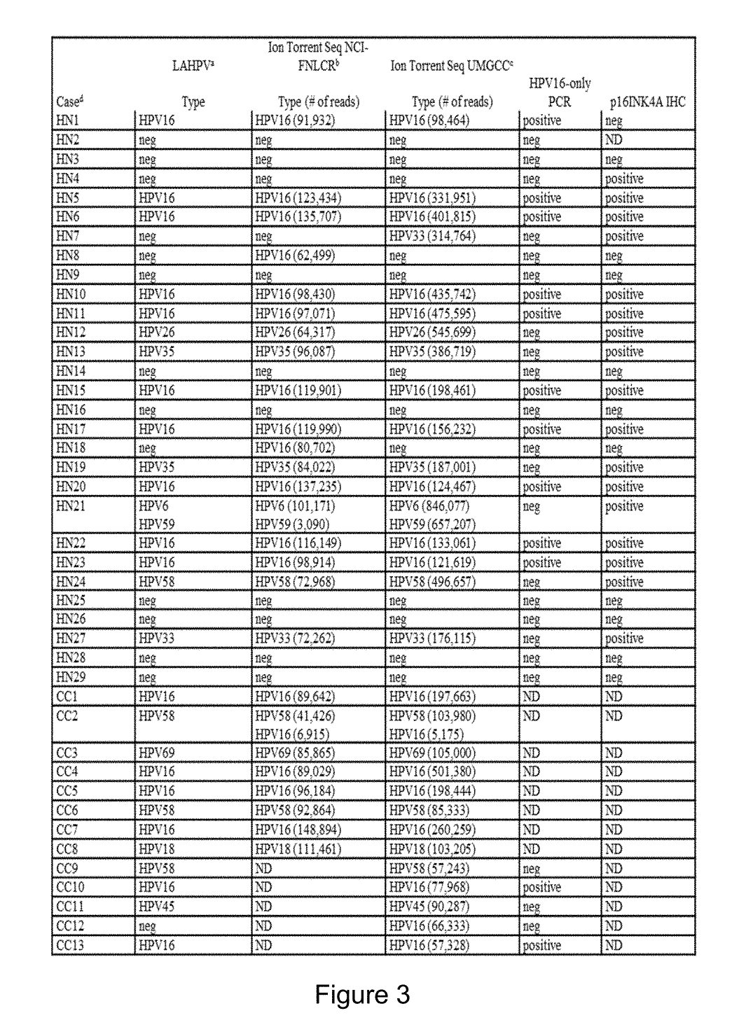

FIG. 3 presents the results from two sequencing sites (NCI-FNLCR and UMGCC) of Ion Torrent NGS HPV Genotyping of genomic DNA from FFPE tumors. All samples were genotyped by LAHPV for comparison. HPV16 DNA PCR and p16 immunostaining results for most cases were also available and are included for reference.

The 29 HNSCC samples comprised both HPV negative cases and cases positive for 7 different HPV genotypes (6, 16, 26, 33, 35, 58, 59) according to LAHPV genotyping. Among the 13 cervical cancer cases, 5 HPV genotypes were represented (16, 18, 45, 58, 69). Although all cervical cancers can be presumed to be HPV positive, LAHPV did not detect HPV in one case (CC12).

A comparison of the results of LAHPV with the UMGCC sequencing site showed concordant results in 28 of 29 HNSCC samples (97%) and 11 out of 13 cervical samples (85%). The discordant HNSCC sample (HN7) was p16 positive by IHC, HPV33 positive by Ion Torrent NGS Genotyping, but negative by LAHPV. One discordant cervical case, CC12, was HPV16 positive by sequencing, where no HPV was detected by LAHPV. These two samples were repeated in triplicate by sequencing, and all three times yielded HPV33 positive (HN7) and HPV16 positive (CC12). The other discordant cervical case had both HPV 58 (103,980 reads) and HPV16 (5,175 reads) detected by NGS genotyping, while LAHPV detected only HPV 58. Sequencing CC2 in triplicate yielded similar results, observing both HPV58 and HPV16 sequences in this sample.

A comparison between the results of LAHPV with sequencing data from NCI-FNLCR showed strong concordance (34 out of 37). Sequencing of cervical sample CC2 by NCI-FNLCR revealed the same co-infected genotypes as described above, HPV58 and HPV16. Two cases (HN8 and HN18) were found to be HPV16 positive by NGS genotyping at NCI-FNLCR, while these same samples were HPV negative by LAHPV, HPV negative by NGS genotyping at UMGCC, p16 negative by IHC, and were HPV16 negative by E6/E7 PCR. The DNA aliquots tested at NCI-FNLCR for HN8 and HN18 were subsequently tested in triplicate at UMGCC and found to be negative for HPV16 in all three replicates. Without being bound to a particular theory, the positive results observed at NCI-FNLCR for HN8 and HN18 are consistent with the explanation that the results represent contaminants introduced into the sequencing reaction from a source other than the sample aliquots provided, and do not represent a false-positive result.

Comparing the sequencing results at the UMGCC and NCI-FNLCR labs, again, 34 of 37 cases (92%) were concordant. This included the two contaminants identified above (HN8 and HN18) and one sample, HN7, which was determined to be HPV negative at NCI-FNLCR and HPV33 positive at UMGCC. This sample failed to produce a ˜150 bp band at NCI-FNLCR and therefore was not included in the sequencing reaction. However, UMGCC observed a faint ˜150 bp for this sample and upon sequencing revealed an HPV 33 genotype. Repeating this sample using the DNA aliquot from NCI-FNLCR confirmed the HPV33 genotype for this sample.

Twenty-eight out of 29 HNSCC samples had data available for p16 overexpression. Concordance between HPV detection and p16 expression was 93% (26/28) when HPV was detected by NGS genotyping at UMGCC, 89% (25/28) by LAHPV and 82% (23/28) by NGS genotyping at NCI-FNLCR. In case HN1, all DNA testing methods including PCR for HPV16 E6/E7 detected HPV16 while p16 was negative, indicating that this tumor is likely a true HPV/p16 discordant case where p16 expression has been lost.

Both the sequencing assay and LAHPV were highly sensitive. A known positive case was used to explore the sensitivity of both assays. In a serial dilution of starting DNA (10, 5, 2.5, 1.25 and 0.625 ng), HPV16 was detected by Ion Torrent NGS Genotyping with as little as 1.25 ng of DNA (obtaining 263,813 reads). The detection limit for this sample in the LAHPV assay was 2.5 ng of DNA.

A large cohort of cervical cancer cases was tested by Ion Torrent NGS Genotyping at NCI-FNLCR (Lou et al., Clin Cancer Res, 2015, 21:5360-5370); the genotypes detected were not previously reported. Twenty HPV genotypes were detected in this cohort, including all 13 high-risk genotypes (16, 18, 31, 33, 35, 39, 45, 51, 52, 56, 58, 59, and 68). Types 6, 26, 44, 53, 67, 69 and 81 were also detected.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for detecting HPV nucleic acid in a biological sample of a subject suspected of having an HPV infection or an HPV associated disease, the method comprising: (a) isolating a nucleic acid sample from a biological sample obtained from the subject; (b) determining the nucleic acid sequence of the nucleic acid sample using a next generation (Next Gen) sequencing assay; and (c) determining the genotype of HPV in the sample,
wherein the method of determining the nucleic acid sequence of the nucleic acid sample comprises: (i) contacting the nucleic acid sample with at least one forward PCR primer, (ii) contacting the nucleic acid sample with at least one reverse PCR primer, (iii) amplifying the HPV nucleic acid using PCR, and (vi) sequencing the amplified nucleic acid products using a Next Gen Sequencer, and
wherein the at least one forward PCR primer comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further comprises a nucleic acid sequence for use as a sequencing adaptor.

2. (canceled)

3. (canceled)

4. The method of claim 1, wherein the at least one forward PCR primer comprises at least two forward PCR primers, wherein each of the at least two forward PCR primers comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further wherein each of the at least two forward PCR primers further comprises a nucleic acid sequence for use as a sequencing adaptor.

5. The method of claim 1, wherein the at least one forward PCR primer comprises at least nine forward PCR primers, wherein each of the at least nine forward PCR primers comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further wherein each of the at least nine forward PCR primers further comprises a nucleic acid sequence for use as a sequencing adaptor.

6. The method of claim 1, wherein the sequencing adaptor comprises or consists of the sequence set forth in SEQ ID NO:10.

7. The method of claim 1, wherein the at least one reverse PCR primer comprises a nucleic acid sequence selected from the group comprising SEQ ID NO:11, SEQ ID NO:12, and SEQ ID NO:13, and wherein each of the at least one reverse PCR primers comprises a nucleic acid sequence for use as a barcode and a nucleic acid sequence for use as a sequencing adaptor.

8. The method of claim 1, wherein the at least one reverse PCR primer comprises at least two reverse PCR primers, wherein each of the at least two reverse PCR primers comprises a nucleic acid sequence selected from the group comprising SEQ ID NO:11, SEQ ID NO:12, and SEQ ID NO:13, further wherein each of the at least two reverse PCR primers comprises a nucleic acid sequence for use as a barcode and a nucleic acid sequence for use as a sequencing adaptor.

9. The method of claim 1, wherein the at least one reverse PCR primer comprises at least three reverse PCR primers, wherein at least one of the reverse PCR primers comprises the nucleic acid sequence set forth in SEQ ID NO:11, at least one of the PCR primers comprises the nucleic acid sequence set forth in SEQ ID NO:12, and at least one of the PCR primers comprises the nucleic acid sequence set forth in SEQ ID NO:13, further wherein each of the at least one reverse PCR primers comprises a nucleic acid sequence for use as a barcode and a nucleic acid sequence for use as a sequencing adaptor.

10. The method of claim 7, wherein the sequencing adaptor comprises or consists of the sequence set forth in SEQ ID NO: 17.

11. (canceled)

12. (canceled)

13. (canceled)

14. The method of claim 1, wherein the method of detecting HPV nucleic acid in the subject comprises aligning a set of sequence data reads to at least one HPV reference sequence, identifying sequencing reads that align to the at least one HPV reference sequence, evaluating the nucleic information of the identified sequencing reads to identify at least one HPV subtype-specific genetic variation, and determining the subtype of the HPV based on the at least one HPV subtype-specific genetic variations.

15. (canceled)

16. The method of claim 14, wherein multiple HPV subtypes are determined for a sample from a subject.

17. (canceled)

18. (canceled)

19. The method of claim 1, wherein the method of detecting HPV nucleic acid in the subject comprises aligning a set of sequence data reads to at least one HPV reference sequence, identifying sequencing reads that align to the at least one HPV reference sequence, evaluating the nucleic information of the identified sequencing reads to identify at least one HPV genotype.

20. (canceled)

21. The method of claim 19, wherein multiple HPV genotypes are determined for a sample from a subject.

22. The method of claim 19, wherein multiple HPV genotypes are determined when at least 1% of the sequencing reads for a sample carry a genotype specific nucleic acid.

23. (canceled)

24. The method of claim 1, further comprising (f) diagnosing the subject with infection by HPV.

25. The method of claim 24, further comprising (g) providing a treatment to the subject on the basis of the diagnosis.

26. (canceled)

27. (canceled)

28. (canceled)

29. (canceled)

30. A composition comprising at least one PCR primer, wherein the at least one PCR primer comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further comprises a nucleic acid sequence for use as a sequencing adaptor.

31. The composition of claim 30, wherein the nucleic acid sequence for use as a sequencing adaptor consists of the sequence set forth in SEQ ID NO:10.

32. A composition comprising at least one PCR primer wherein the at least one PCR primer comprises a nucleic acid sequence selected from the group comprising SEQ ID NO: 11, SEQ ID NO:12 and SEQ ID NO:13 and further comprises a nucleic acid sequence for use as a barcode and a nucleic acid sequence for use as a sequencing adaptor.

33. The composition of claim 32, wherein the nucleic acid sequence for use as a sequencing adaptor consists of the sequence set forth in SEQ ID NO: 17.

34. (canceled)

35. A kit comprising:
a) a first composition comprising at least one PCR primer, wherein the at least one PCR primer comprises a nucleic acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9 and further comprises a nucleic acid sequence for use as a sequencing adaptor,
and
b) a second composition comprising at least one PCR primer wherein the at least one PCR primer comprises a nucleic acid sequence selected from the group comprising SEQ ID NO: 11, SEQ ID NO:12 and SEQ ID NO:13 and further comprises a nucleic acid sequence for use as a barcode and a nucleic acid sequence for use as a sequencing adaptor,
for use in the method of claim 1.

36. The kit of claim 35, further comprising control primers.

37. The kit of claim 36, wherein the control primers have nucleic acid sequences as set forth in SEQ ID NO:27 and SEQ ID NO:28.
